(19) United States
(12) Patent Application Publication
(10) Pub. No.: US 2015/0014541 A1
(43) Pub. Date: Jan. 15, 2015

(54) Method for measuring the concentration of a gas component in a measuring gas

(71) Applicant: Siemens Aktiengesellschaft, Muenchen (DE)

(72) Inventors: Daniel Depenheuer, Schwegenheim (DE); Christoph Wolfgang Marquardt, Karlsruhe (DE)

(73) Assignee: Siemens Aktiengesellschaft

(21) Appl. No.: 14/324,443

(22) Filed: Jul. 7, 2014

(30) Foreign Application Priority Data
Jul. 9, 2013 (DE) 10 2013 213 458.4

(51) Int. Cl. G01N 21/59; G01N 33/00

(52) U.S. Cl. CPC G01N 21/59; G01N 33/0062; G01N 2201/06113
USPC 250/341.1

(57) ABSTRACT

A method for measuring the concentration of a gas component in a measuring gas, wherein a semiconductor laser is periodically actuated by a current ramp to scan a selected absorption line in a wavelength-dependent manner and to determine the concentration of the gas component based on the reduction in the light intensity as a result of the absorption of the light at the location of the absorption line.

### BACKGROUND OF THE INVENTION

1. Field of the Invention

The invention relates to a method for measuring the concentration of a gas component in a measuring gas by virtue of the intensity of the light of a wavelength-tunable semiconductor laser being detected after passing through the measuring gas and the concentration of the gas component being determined based on the reduction in the light intensity as a result of the absorption of the light at the location of a selected absorption line of the gas component, wherein the semiconductor laser is periodically actuated by a current ramp in order to scan the absorption line of the gas component in a wavelength-dependent manner, the semiconductor laser is actuated by a first current signal in a first phase immediately before the current ramp and/or by a second current signal in a second phase immediately after the current ramp and the light intensity detected at the location of the absorption line is normalized by light intensities detected in the first and second phase.

2. Description of the Related Art

EP 2 072 979 A1 or DE 10 2011 080 086 A1 discloses a method for measuring the concentration of a gas component in a measuring gas.

DE 10 2012 202 893 B3 likewise discloses the practice of using a burst signal for normalizing the light intensity detected at the location of the absorption line.

DE 10 2011 079 342 B3 discloses the practice of actuating the semiconductor laser, in a phase inserted between a first and a subsequent second current ramp, by a constant current corresponding to the start value of the second current ramp. When the first current ramp is modified, the duration of the constant current phase is modified such that the amount of current supplied to the laser by the first current ramp and the constant current remains the same.

When passing through the measuring gas, a small portion of the light is absorbed in a wavelength-dependent manner by the infrared-active gas components of the measuring gas. Additionally, there is absorption by optical components, e.g., windows, in the light path and by aerosols, such as smoke particles; this absorption is wavelength-independent in the small wavelength regions of interest. Therefore, it is necessary to normalize the measurement to free it from interfering components as a result of the wavelength-independent absorption. In the known methods, the normalization is achieved with the aid of a first and/or a second current signal in the form of a current burst, in which the current repeatedly alternates with a burst frequency between zero and a maximum value. The maximum value of the first current burst corresponds to the start value of the current ramp and the maximum value of the second current burst corresponds to the final value of the current ramp, and so the wavelength of the light generated at the locations of the current bursts comes to rest outside of the wavelength regions of the absorption lines of the gas components to be measured and of other infrared-active gas components in the measuring gas. The light intensity detected at the location of the absorption line can be normalized by division by the light intensity detected at the location of a current burst or by a light intensity value calculated by interpolation of the light intensities detected at the locations of the two current bursts.

Since each measured spectrum needs to be normalized, each measurement cycle contains at least one current burst in addition to the current ramp. Switching the semiconductor laser on and off causes a quickly and strongly varying thermal load or heat generation rate. Since the loss power of the laser increases more than proportionally with the current, this in turn leads to a temperature of the laser varying nonlinearly with time. The duration of this temperature response to switching on and off may vary strongly depending on laser design and installation type (thermal coupling). Thus, several 10 to 100 ms may be necessary for the semiconductor laser to return to a stable thermal state. Since the generated wavelength substantially depends on the temperature of the laser, the wavelength likewise varies strongly, in a long-lasting manner and nonlinearly in time. Depending on the type of laser, this behavior can influence the measurement so strongly that measurement operation using this semiconductor laser is impossible. The strong change in the laser temperature as a result of the current burst is expressed not only in an instability of the wavelength, but possibly also in the optical power; i.e., the optical power is significantly higher after each switching-on of the semiconductor laser, i.e., at the start of an individual burst pulse, than at the end of the burst pulse. This can be explained by the strong temperature increase after switching on the laser, as a result of which the optical power of the laser reduces in the case of the same diode current. As a result, the light intensity detected at the locations of the current burst or bursts may be strongly afflicted with errors, depending on the laser type.

In order to solve the problem, provision can be made for waiting times that are as long as possible after each current burst so as to give the semiconductor laser time to return to a stable thermal state. As mentioned previously, several 10 to 100 ms may be necessary for this, depending on the laser, and so conventional measurement rates in the range from 10 to 100 Hz cannot be achieved.

Furthermore, it is possible to restrict oneself to semiconductor lasers in which the problem manifests itself as little as possible. This may contain both the selection of a fitting laser type and an individual selection of the lasers; however, this contains, in part, great restrictions in the laser specifications and much complexity in the laser screening.

Finally, the problem can be ignored, but this would have a more or less strong influence on the measurement power, depending on the laser.

### SUMMARY OF THE INVENTION

It is therefore an object of the invention to directly compensate for the above-described effect of changes, due to age or any other reasons, in the intensity of generated light on measurement of the concentration of a gas component in a measuring gas.

This and other objects and advantages are achieved in accordance with the invention by virtue of the fact that, in the method of the type set forth at the outset, the first current signal consists of a constant current corresponding to the start value of the current ramp, the second current signal consists of a constant current corresponding to the final value of the current ramp, the semiconductor laser is switched to a no current state during a third phase after a predetermined number of a plurality of current ramps and the light intensities detected in each case most recently in the first and/or second and in the third phase are used for normalizing the light intensity detected at the location of the absorption line.

The normalization is performed by measuring the light intensity when the semiconductor laser is switched on and when it is switched off. However, this no longer occurs directly in succession, but rather it is only still the light intensity when the laser is switched on that is measured in each measurement cycle, i.e., in a phase with constant current. At regular intervals, i.e., after a predetermined number of measurement cycles, a zero spectrum with the laser switched off is measured instead of a normal spectrum. As a result, the necessary information about the light intensity when the semiconductor laser is switched on and off is available, as in the case of normalization with the current bursts. The measured light intensity when the laser is switched off is determined by three components (i) the dark current of the employed detector which, in the case of a photodiode, is substantially determined by the temperature thereof, which normally does not change very quickly and can optionally be stabilized by a Peltier element, (ii) the thermal radiation from the surroundings, which typically only changes slowly and (iii) light from other sources than the semiconductor laser.

The cross sensitivity in relation to interfering light sources can advantageously be reduced by virtue of the spectral sensitivity being restricted by, e.g., a narrow-band transmission filter.

In order to interrupt the sequence of measurement cycles as little as possible by switching off the semiconductor laser, the frequency of the zero-current phase can be adapted to the change in the measured zero spectrum or the calculated transmission. That is, the light intensities currently detected in the constant current phases (first and/or second phases) and/or in the zero-current phase (third phase) are compared to the light intensities previously detected in the same phases and the predetermined number of current ramps or measurement cycles between the zero-current phases is increased or reduced depending on the magnitude of the changes in the detected light intensities.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the following text, the invention will be explained on the basis of examples, with reference being made to the figures in the drawing in which:

FIG. 1 shows a schematic illustration of a laser spectrometer for performing the method in accordance with the invention;

FIGS. 2 to 4 show different examples for actuating the semiconductor laser; and

FIG. 5 is a flowchart of the method in accordance with the invention.

### DETAILED DESCRIPTION OF THE PREFERRED EMBODIMENTS

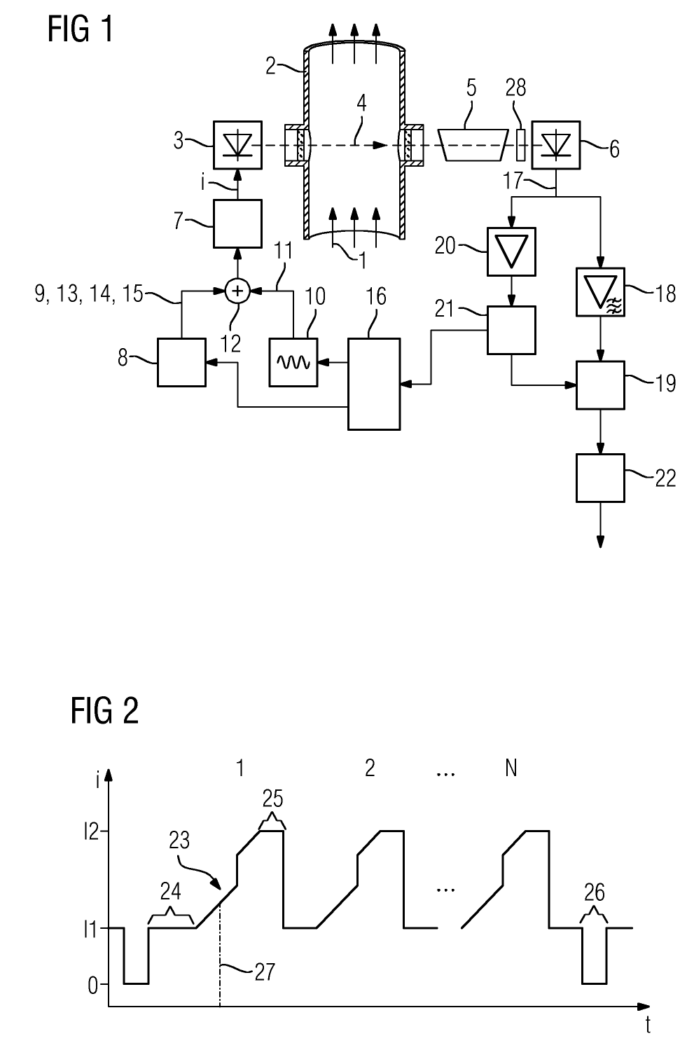

FIG. 1 shows a laser spectrometer for measuring the concentration of at least one gas component of interest in a measuring gas 1, which is contained in a measurement volume 2, e.g., which in this case flows through a process gas line. The spectrometer contains a semiconductor laser 3, in this case a laser diode, the light 4 of which passes through the measuring gas 1 and optionally through a downstream reference gas cuvette 5, filled with a reference gas, and is incident on a detector 6. The semiconductor laser 3 is actuated with an injection current i by a controllable current source 7, where the intensity and wavelength of the generated light 4 depend on the current i and the operating temperature of the semiconductor laser 3.

The current source 7 is actuated periodically with a ramp-shaped function 9 by a first signal generator 8, in order both to vary the current i through the semiconductor laser 3 (current ramp) and to scan a selected absorption line of the gas component of interest in the measuring gas 1 in a wavelength-dependent manner using the correspondingly modulated light 4. A second signal generator 10 generates a sinusoidal signal 11 with the frequency f, by which the ramp-shaped function 9 is modulated in an adding member 12. In each measurement period, the first signal generator 8 furthermore generates a control signal 13 or 14 in a first actuation phase immediately before the ramp-shaped function 9 and/or in a second phase after the ramp-shaped function 9 to set the current i to the start value of the current ramp for the duration of the first phase and to set the current to the final value of the current ramp for the duration of the second phase. Furthermore, the first signal generator 8 generates a control signal 15 after a predetermined number of a plurality of current ramps to switch the semiconductor laser 3 to no current during a third phase. The succession in time of the signals 11 to 15 is controlled by a control apparatus 16.

The wavelength of the light 4 generated during the actuation of the laser 3 with the start and/or final value of the current ramp lies outside of the wavelength regions of the gas component to be measured and of other infrared-active gas components of the measuring gas 1. When passing through the measuring gas 1, a small portion of the light 4 generated by the laser 3 is absorbed by the infrared-active gas components of the measuring gas 1 in a wavelength-dependent manner. Additionally, there is a wavelength-independent absorption by optical components, such as windows, in the light path and by aerosols, such as smoke particles.

Due to the actuation of the laser 3 with the current ramp, the wavelength of the generated light 4 is modified periodically within a tuning range and, in the process, the selected absorption line of the gas component of interest is scanned in a wavelength-dependent manner. While the laser 3 is tuned, the wavelength of the light 4 is modulated at the same time with the frequency f due to the signal 11. When scanning the absorption line, a small portion of the light 4 is absorbed thereby. Depending on the detected light intensity I, the detector 6 generates a detector signal 17, the second harmonic of which (2f-signal component) is amplified in a frequency-selective amplifier 18 and fed to a normalization stage 19. The signal portions of the detector signal 17 resulting from the actuation of the current source 7 (and hence of the laser 3) with the control signals 13, 14, 15 are amplified in a further amplifier 20. In a computer 21 arranged downstream, an intensity value at the position of the absorption line is calculated from the detected light intensities, which intensity value would be measured there in the case of a non-existent absorption line. Using this intensity value, the light intensity detected in the form of the 2f-signal component of the detector signal 16 at the location of the absorption line 15 is normalized in the normalization stage 19. The 2f-signal component of the detector signal 17 thus normalized is processed further in a downstream evaluation apparatus 22 and evaluated for establishing the concentration of the gas component of interest in the measuring gas 1.

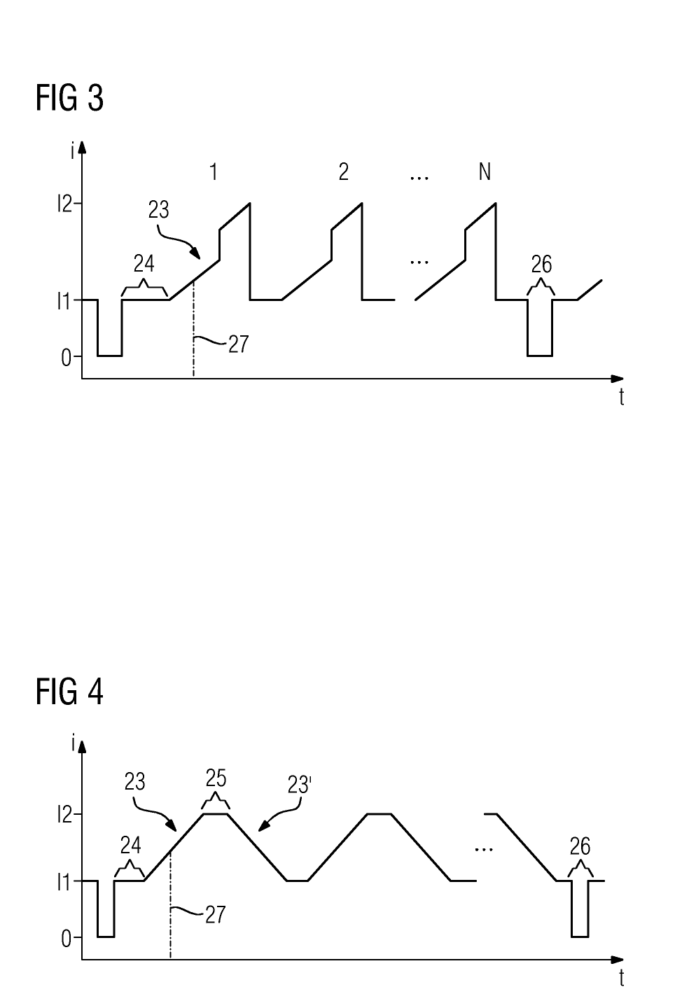

FIG. 2 shows a first example for the profile of the current i for actuating the semiconductor laser 3. The wavelength of the generated light 4 is modified within a tuning range via periodically generated current ramps 23 and the absorption line of the gas component of interest is scanned in successive measurement cycles. As shown, the current ramp 23 can be subdivided into two sections with different current profile where, in one section, the absorption line of the gas component of interest and, in the other section, an absorption line of the reference gas in the reference gas cuvette 5 is scanned. In each measurement cycle, the laser 3 is actuated in a first phase 24 immediately before the current ramp 23 by a constant current I1 corresponding to the start value of the current ramp 23 and in a second phase 25 immediately after the current ramp 23 by a constant current I2 corresponding to the final value of the current ramp 23. A third phase 26, in which the semiconductor laser 3 is switched off, in each case follows after a predetermined number N of measurement cycles.

The example shown in FIG. 3 differs from that shown in FIG. 2 in that there is no second phase 25 with the constant current I2. Alternatively, provision can also be made only for the first phase 24 with the constant current I1.

In the example shown in FIG. 4, a falling current ramp 23′ follows each increasing current ramp 23, and so the start value I1 of the increasing current ramp 23 corresponds to the final value I1 of the falling current ramp 23′ and the start value 12 of the falling current ramp 23′ corresponds to the final value I2 of the increasing current ramp 23′.

The light intensity detected in each measurement cycle at the location 27 of the absorption line can be normalized based on the light intensity detected in the phases 24 and 25, while the latter is normalized, in turn, by the light intensity detected in phase 26.

Measuring the light intensity during the third phase, when the laser 3 is switched off, can be influenced by a cross sensitivity of the laser spectrometer in relation to interfering light sources. As shown in FIG. 1, this influencing can be reduced by a narrow-band transmission filter 28 in the light path.

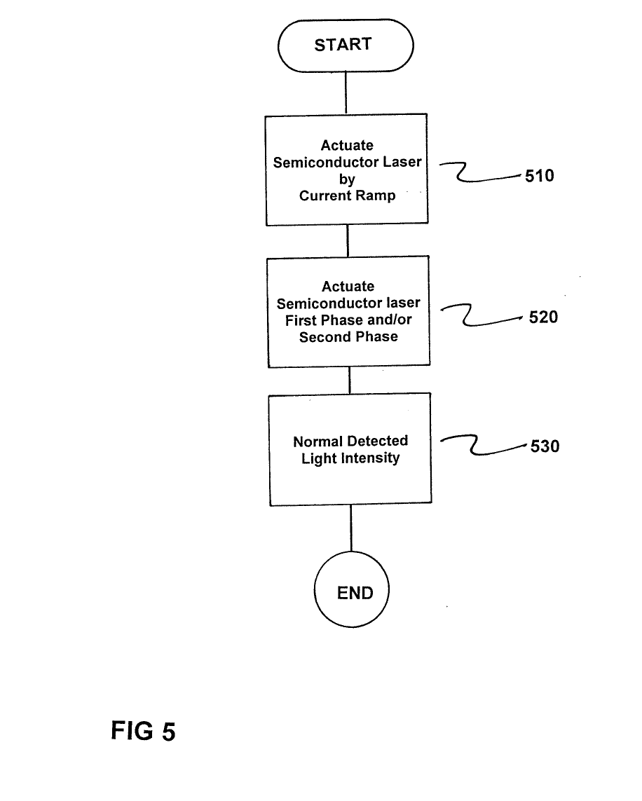

FIG. 5 is a flowchart of a method for measuring concentration of a gas component in a measuring gas (1) by virtue of an intensity of light (4) of a wavelength-tunable semiconductor laser (3) being detected after passing through the measuring gas (1) and the concentration of the gas component being determined based on the reduction in the intensity of the light as a result of absorption of the light (4) at the location of a selected absorption line (27) of the gas component.

The method comprises actuating the semiconductor laser (3) periodically by a current ramp (23) to scan the absorption line (27) of the gas component in a wavelength-dependent manner, indicated in step 510.

Next, the wavelength-tunable semiconductor laser (3) is actuated in a first phase (24) either immediately before the current ramp (23) by a first current signal and/or in a second phase (25) immediately after the current ramp (23) by a second current signal, as indicated in step 520.

The light intensity detected at the location of the absorption line (27) is now normalized by light intensities detected in either the first and/or the second phases (24, 25), as indicated in step 530.

In accordance with the method of the invention, the first current signal consists of a constant current (I1) corresponding to a start value of the current ramp (23), the second current signal consists of a constant current (I2) corresponding to the final value of the current ramp (23), the semiconductor laser (3) is switched to no current during a third phase (26) after a predetermined number (N) of a plurality of current ramps (23), and each detected light intensity most recently in either the first, second and/or third phases (24, 25, 26) is used to normalize the light intensity detected at the location of the absorption line (27).

While there have been shown, described and pointed out fundamental novel features of the invention as applied to a preferred embodiment thereof, it will be understood that various omissions and substitutions and changes in the form and details of the methods described and the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1-3. (canceled)

4. A method for measuring concentration of a gas component in a measuring gas by virtue of an intensity of light of a wavelength-tunable semiconductor laser being detected after passing through the measuring gas and the concentration of the gas component being determined based on a reduction in the intensity of the light as a result of absorption of the light at a location of a selected absorption line of the gas component, the method comprising:
actuating the semiconductor laser periodically by a current ramp to scan the absorption line of the gas component in a wavelength-dependent manner;
actuating the wavelength-tunable semiconductor laser in a first phase at least one of (i) immediately before the current ramp by a first current signal and (ii) in a second phase immediately after the current ramp by a second current signal; and
normalizing the light intensity detected at the location of the absorption line by light intensities detected in at least one of (i) the first phase and (ii) the second phase;
wherein the first current signal consists of a constant current corresponding to a start value of the current ramp;
wherein the second current signal consists of a constant current corresponding to the final value of the current ramp;
wherein the semiconductor laser is switched to a no current state during a third phase after a predetermined number of a plurality of current ramps; and
wherein each detected light intensity most recently in at least one of (i) the first phase, (ii) the second and (iii) the third phase is used to normalize the light intensity detected at the location of the absorption line.

5. The method as claimed in claim 4, wherein light intensities currently detected in at least one of (i) the first phase, (ii) the second phase and (ii) the third phase are compared to light intensities previously detected in the same phases, and wherein the predetermined number of current ramps between third phases is one of (i) increased and (ii) reduced depending on a magnitude of changes in the detected light intensities.

6. The method as claimed in claim 4, wherein a narrow-band transmission filter is utilized to reduce the influence of interfering radiation on the detection.
